(19) United States
(12) Patent Application Publication
(10) Pub. No.: US 2016/0199351 A1
(43) Pub. Date: Jul. 14, 2016

(54) COMPOSITIONS AND METHODS FOR INHIBITING FUNGAL INFECTIONS

(71) Applicants: ARNO THERAPEUTICS, INC., Flemington, NJ (US); OHIO STATE INNOVATION FOUNDATION, Columbus, OH (US)

(72) Inventors: Chad A. Rappleye, Powell, OH (US); Alexander Zukiwski, Clarksburg, MD (US); Stefan Proniuk, Austin, TX (US)

(21) Appl. No.: 14/990,594

(22) Filed: Jan. 7, 2016

Related U.S. Application Data
(60) Provisional application No. 62/102,436, filed on Jan. 12, 2015.
(60) Provisional application No. 62/143,777, filed on Apr. 6, 2015.

(51) Int. Cl. A61K 31/415; A61K 45/06

(52) U.S. Cl. CPC A61K 31/415; A61K 45/06

(57) ABSTRACT

Compositions and methods for inhibiting fungal growth by administering AR-12 to a host infected with a fungus such as Histoplasma capsulatum, Aspergillus fumigatus, and Trichophyton rubrum, Paecilomyces, Rhizopus, Fusarium, Scedosporium, Lomentospora, Apophysomyces, Coccidioides, Blastomyces, non-albicans Candida, and Pneumocytis are provided.

### PRIORITY CLAIM

This application claims priority to U.S. Provisional Patent Application Ser. No. 62/102,436 filed on Jan. 12, 2015 and U.S. Provisional Patent Application Ser. No. 62/143,777 filed on Apr. 6, 2015. The above referenced applications are incorporated herein by reference as if restated in full. All references cited herein, including, but not limited to patents and patent applications, are incorporated by reference in their entirety.

### BACKGROUND

Fungal pathogens cause a wide variety of diseases ranging from pulmonary and systemic diseases (e.g., histoplasmosis, invasive candidiasis and aspergillosis) to skin and nail infections (e.g., onychomycosis). Aspergillus causes a variety of pulmonary infections including allergic bronchopulmonary aspergillosis (ABPA), allergic aspergillus sinusitis, aspergilloma, and chronic pulmonary aspergillosis. Histoplasma capsulatum causes a respiratory disease in both immunocompromised as well as immunocompetent individuals. In some individuals, including those with suppressed T-cell function, Histoplasma causes progressive disseminated disease which is fatal if untreated. Fungal pathogens also cause pneumocystis, coccidioidomycosis (e.g., San Joaquin Valley Fever), and blastomycosis.

Candida are small (4-6 μm) thin walled ovoid yeasts which reproduce by budding. Candida organisms appear in three forms in tissues; blastospores, pseudohyphae and hyphae. The genus Candida contains more than 150 species, however only a few cause disease in humans. Candida infections can be classified as 1) Mucocutaneous, or 2) Invasive. Mucocutaneous candidiasis can affect the skin, oral pharynx, esophageal and vulvovaginal areas. Mucocutaneous infections are common in all climates. Vulvovaginal candidiasis is one of the most common genital problems of women in both industrialized and developing countries. Extensive use of antibiotics, development of human immunodeficiency virus (HIV) infection, the increasing prevalence of diabetes mellitus, and local genital immune factors are all contributors to the widespread prevalence of vulvovaginal candidiasis. Oropharyngeal and esophageal candidiasis are typically encountered in association with local mucosal injury or as a result of defects in cell mediated immunity.

Invasive candidiasis is an opportunistic infection caused by a number of Candida fungal species including C. albicans, C. guilliermondii, C. krusei, C. parapsilosis, C. tropicalis, C. kefyr, C. lusitaniae, C. dubliniensis and C. glabrata. Fifty percent of Candida infections are caused by non-albicans Candida. The more severe infections caused by Candida species have been described in the literature as deeply invasive candidiasis, invasive candidiasis or disseminated candidiasis. These life threatening infections are caused by candida species invading the blood stream (candidemia) or by invading deep seated organs. Host factors are very important in the development of candidemia and deep seated candidiasis, as these infections mainly occur in debilitated patients. Invasive candidiasis is most often found in severely ill patients, such as those patients hospitalized in intensive care units [ICU] or those patients with neutropenia. [Blot 2002, Blot 2008, Darouiche 2009]. These invasive infections can result via infection from candida organisms through superficial oesophageal erosions, joint or deep wound infections from contiguous spread of the organisms from the skin, gallbladder infections from retrograde migration of gut flora, kidney infections resulting from urinary catheter use and peritoneal spread from gastrointestinal tract perforations. However, the most common invasive candidiasis is a result of haematogenous seeding as a complication of candidemia. [Edwards 2012]. The portal of entry 80% of the cases of candidemia arise from the use of vascular access devices, including central venous catheters, haemodialysis catheters and implanted ports [Brusselaers 2011].

Histoplasma is a dimorphic fungal pathogen found in the United States primarily along the Ohio and Mississippi river valleys. It grows as an environmental mold, producing conidia which are the infectious form. Infection is due to inhalation of the conidia which differentiate into pathogenic yeasts upon exposure to mammalian body temperatures. Within the host, Histoplasma yeast parasitize macrophages of the immune system and disseminates to extrapulmonary sites via the reticuloendothelial system.

Trichophyton is a filamentous fungus which grows as hyphae in and on host tissues. The fungus is acquired by contact with material contaminated with Trichophyton hyphae and hyphal elements (e.g., often by contaminated shed skin scales). The hyphae produce keratinases which enable them to use keratin as a nutrient source. Trichophyton colonizes the keratinized stratum corneum, presenting a chronic source of continued infection. Although direct invasion of living tissue is rare, the presence of the fungus can induce inflammatory responses in the surrounding tissue. Disease conditions caused by Trichophyton include infections of the skin (e.g., tinea pedis (athlete's foot) and tinea corporis (ringworm)), and of the nails and nail bed (tinea unguium or onychomycosis).

Current treatments for fungal pathogens include clotrimazole, econazole, ketoconazole, miconazole, tioconazole, fluconazole, posaconazole, itraconazole, voriconazole, isavuconazonium, terbinafine, nystatin, amorolfine, griseofulvin, caspofungin, micafungin, anidulafungin, tevaborole, efinaconazole, amphotericin B deoxycholate and liposomal amphotericin B, and are typically provided topically, orally, or intravenously. Side-effects include liver damage, allergic reactions, and hormonal effects. In particular, triazole-based drugs have significant host side-effects such as reversible increases in hepatic enzymes, nausea, vomiting, diarrhoea, abdominal pain, constipation, dyspepsia, allergic reactions (e.g., pruritus), rash, urticarial, angioedema, and hepatitis after prolonged use. In addition, echinocandin-based drugs are not effective against pathogenic-phase of Histoplasma capsulatum.

AR-12 (a.k.a. OSU-03012) has been previously shown to exhibit anti-tumor and anti-bacterial activity. It is thought that AR-12 induces autophagy of cells harboring intracellular bacteria. While Krysan, et al. (US Patent Application Publication 2012/0122872) demonstrated the activity of AR-12 (OSU-03012) with respect in certain fungal species (Candida albicans and Cryptococcus neoformans), the antifungal activity of AR-12 has not been demonstrated with respect to a wide range of fungal species or sub-species, and the precise antifungal mechanism of AR-12 has not been shown.

### SUMMARY

Aspects described herein provide methods and composition for inhibiting fungal infections in a host. As described herein, AR-12 can be administered to fungus or fungal cells to inhibit or reduce the growth of fungus. In another aspect, AR-12 can be administered to a mammal infected with a fungus to inhibit or reduce the growth of the fungus or to treat a condition caused by the fungus. The route of administration for AR-12 can be any suitable route used for current antifungal treatments (e.g., topical, oral, ophthalmic, intravenous, intranasal, inhalation, transdermal).

In another aspect, the fungus is selected from the group consisting of Histoplasma capsulatum, Aspergillus fumigatus, and Trichophyton rubrum. In another aspect, AR-12 can penetrate into or permeate into a nail infected with a nail fungus (e.g., Trichophyton species). In a further aspect, AR-12 can permeate through the nail. In yet another aspect, permeation enhancer (e.g., PEG400 or surfactants) can be used to enhance permeation of the infected nail by AR-12.

Further aspects provide methods for inhibiting the growth of a fungus comprising administering an amount of AR-12 to a host infected with a fungus sufficient to achieve at least about 50% inhibition of fungal growth (MIC50). In another aspect, AR-12 is provided in an amount sufficient to achieve at least about 90% growth inhibition of fungal growth (MIC90). In this aspect, AR-12 can be provided in an amount from about 10 μM to about 20 μM (micromolar) or about 10 μM to about 40 μM for example, to inhibit the growth of or kill Histoplasma capsulatum or Aspergillus fumigatus. In another aspect, AR-12 can be provided in an amount from about 8 μM to about 16 μM or about 8 μM to about 24 μM inhibit or kill Trichophyton cells. In another aspect, AR-12 can be provided in an amount of at least about 3 μM to inhibit growth of fungal cells.

Yet further aspects provide methods for inhibiting the growth of Trichophyton rubrum by administering AR-12 to a nail (e.g., toenail, fingernail or thumbnail) infected with Trichophyton rubrum such that AR-12 can penetrate into the nail and inhibit the growth of the Trichophyton rubrum. In another aspect, AR-12 can penetrate and pass through the infected nail.

In another aspect, the fungus is selected from the group consisting of Paecilomyces, Rhizopus, Fusarium, Scedosporium, Lomentospora, Apophysomyces, Coccidioides, Blastomyces, non-albicans Candida (including C. guilliermondii, C. krusei, C. parapsilosis, C. tropicalis, C. kefyr, C. lusitaniae, C. dubliniensis and C. glabrata), and Pneumocytis. Further aspects provide methods for inhibiting the growth of a fungus selected from the group consisting of Paecilomyces, Rhizopus, Fusarium, Scedosporium, Lomentospora, Apophysomyces, Coccidioides, Blastomyces, non-albicans Candida, and Pneumocytis comprising administering an amount of AR-12 to a host infected with a fungus in an amount sufficient to achieve a concentration in the host (e.g., blood, tissue) that inhibits at least about 50% of fungal growth (MIC50). In another aspect, AR-12 is administered in an amount sufficient to achieve a concentration that inhibits about 100% growth inhibition of fungal growth (MIC100).

In this aspect, AR-12 can be administered to a host in amount sufficient to achieve a concentration in the blood of the host from about 1 μg/ml to about 5 μg/ml (or the equivalent concentration in tissue or an organ) to inhibit the growth of or kill one or more fungi selected, for example, from the group consisting of Paecilomyces, Rhizopus, Fusarium, Scedosporium, Lomentospora, Apophysomyces, Coccidioides, non-albicans Candida, and Blastomyces. In another aspect, AR-12 can be administered to a host in an amount sufficient to achieve a concentration in the blood of the host of at least about 1 μg/ml (or the equivalent concentration in tissue or an organ) to inhibit growth of fungal cells.

In another aspect, methods for inhibiting the growth of the fungus Pneumocytis are provided comprising administering an amount of AR-12 to a host infected with Pneumocytis sufficient to achieve a concentration in the host (e.g., blood, tissue or organ) that inhibits the growth of the Pneumocytis fungus by at least about 50%. In this aspect, AR-12 can be administered to a host in an amount sufficient to achieve concentrations in the blood of the host of, for example, about 4.82 μg/ml, about 18.32 μg/ml, and about 41.3 μg/ml (or the equivalent concentration in tissue or an organ). In another aspect, AR-12 can be administered to a host in amount sufficient to achieve a concentration in the blood of the host from about 1 to about 100 μg/ml, 5 to about 50 μg/ml, or about 10 μg/ml to about 20 μg/ml (or the equivalent concentration in tissue or an organ).

Yet further aspects provide methods of inhibiting the growth of fungi (e.g., mold and yeast forms) by providing AR-12 and at least one additional anti-fungal compound (e.g., clotrimazole, econazole, ketoconazole, miconazole, tioconazole, fluconazole, posaconazole, itraconazole, voriconazole, isavuconazonium, terbinafine, nystatin, amorolfine, griseofulvin, caspofungin, micafungin, anidulafungin, tevaborole, efinaconazole, amphotericin B deoxycholate and liposomal amphotericin B).

### BRIEF DESCRIPTION OF THE DRAWINGS

The feature and nature of the present disclosure will become more apparent from the detailed description set forth below when taken in conjunction with the accompanying drawings.

FIG. 1A is a graph showing an exemplary dose-response curve for the growth of Histoplasma capsulatum after treatment with AR-12;

FIG. 1B is a graph showing viability of Histoplasma capsulatum after treatment with AR-12, fluconazole, or both;

FIG. 1C shows viability staining of Histoplasma capsulatum yeasts following antifungal drug treatment with AR-12 or fluconazole;

FIG. 2A is an exemplary dose-response growth curve for AR-12 treated Aspergillus fumigatus mycelia;

FIG. 2B is a graph illustrating relative mycelia growth following treatment with antifungal agents AR-12, amphotericin, caspofungin, or voriconazole;

FIG. 2C shows exemplary viability staining of Aspergillus fumigatus mycelia following antifungal drug treatment with AR-12 or caspofungin ; and

FIG. 3 is an exemplary dose-response curve for the effect of AR-12 on the growth of Trichophyton rubrum mycelia.

### DETAILED DESCRIPTION

The disclosed methods and compositions below may be described both generally as well as specifically. It should be noted that when the description is specific to an aspect, that aspect should in no way limit the scope of the methods. All references cited herein are hereby incorporated by reference in their entirety.

In one aspect the AR-12 can be administered to a host infected with a fungus (e.g., Histoplasma, Aspergillus, Trichophyton, Paecilomyces, Rhizopus, Fusarium, Scedosporium, Lomentospora, Apophysomyces, Coccidioides, Blastomyces, non-albicans Candida, and Pneumocytis) in an amount sufficient to achieve a concentration in the host sufficient to inhibit the growth of and/or reduce the amount of the fungus.

As used herein, the term “administer” or “administered” refers to applying, ingesting, inhaling or injecting, or prescribing an active ingredient to treat a host or patient in need of treatment. The host can be a mammal (e.g., humans, dogs, cats, horses, cows). As described herein, AR-12 inhibits or kills the fungus (e.g., Histoplasma capsulatum and Aspergillus fumigatus) at low micromolar levels. In another aspect, AR-12 prevents the growth of Trichophyton rubrum.

As used herein, “concentration in the host” refers to a concentration of a drug (e.g., AR-12, an additional anti-fungal drug) in the blood, tissue or organ of the host. Concentration can be expressed, for example, in μM or in μg/ml for liquids or the equivalent for tissue or organs (e.g., μg/m³). In one aspect, the blood, tissue or organ is infected with a fungus.

Aspects described herein provide methods of inhibiting fungal growth in a host infected with a fungus selected from the group consisting of Histoplasma capsulatum, Aspergillus fumigatus, and Trichophyton rubrum by administering AR-12 to the host in an amount sufficient to reduce fungal growth in the host by about 90%. In another aspect, the fungal growth is reduced by about 50%.

In this aspect, AR-12 can be provided to the host in an amount sufficient to achieve a blood, tissue, or organ concentration, for example, between about 8 μM and 24 μM or 10 μM and 40 μM. In this aspect, fungal growth can be inhibited by between about 10% and 50%.

Further aspects provide methods of inhibiting fungal growth in a host infected with one or more fungi selected from the group consisting of Paecilomyces, Rhizopus, Fusarium, Scedosporium, Lomentospora, Apophysomyces, Coccidioides, Blastomyces, non-albicans Candida, and Pneumocytis by administering AR-12 to the host in an amount sufficient to achieve a concentration in the host sufficient to inhibit fungal growth by about 50% or about 100%.

In this aspect, AR-12 can be administered in an amount sufficient to achieve a concentration in the blood of the host between about 1 μg/ml to about 100 μg/ml, or the equivalent concentration in tissue or an organ. In another aspect, the blood concentration is host between about 1 μg/ml to about 16 μg/ml.

Further aspects include administering one or more additional compounds to the host, said one or more additional compounds selected from the group consisting of clotrimazole, econazole, ketoconazole, miconazole, tioconazole, fluconazole, posaconazole, itraconazole, voriconazole, isavuconazonium, terbinafine, nystatin, amorolfine, griseofulvin, caspofungin, micafungin, anidulafungin, tevaborole, efinaconazole, amphotericin B deoxycholate, and liposomal amphotericin B.

Aspects described herein provide methods of inhibiting fungal growth in a host infected with a fungus by administering AR-12 to the host in an amount sufficient to achieve a concentration in the blood of the host between about 1 μg/ml to about 100 μg/ml, or the equivalent concentration in tissue or an organ, for at least about 24, 48, or 72 hours.

Another aspect provides methods of inhibiting fungal growth in a host infected with Pneumocytis by administering AR-12 to the host in an amount sufficient to achieve a concentration in the host to reduce fungal growth in the host by about 50%.

In this aspect, AR-12 can be administered to the host in an amount sufficient to achieve a concentration in the blood of about 4.82 μg/ml, or an equivalent concentration in a tissue or organ. In this aspect, the Pneumocytis can be Pneumocytis carinii.

In this aspect, AR-12 can be administered to the host in an amount sufficient to achieve a concentration in the blood of about 1.78 μg/ml, or an equivalent concentration in a tissue or organ. In this aspect, the Pneumocytis can be Pneumocytis marina.

Further aspects provide methods of inhibiting fungal growth in a host infected with a non-albicans Candida fungus by administering AR-12 to the host in an amount sufficient to achieve a concentration in the host to inhibit non-albicans Candida fungal growth by about 50% or 100%.

In this aspect, AR-12 can be administered in an amount sufficient to achieve a concentration in the host blood between about 1 μg/ml to about 100 μg/ml, or between about 1 μg/ml to about 16 μg/ml or an equivalent concentration in a tissue or organ.

In one aspect, AR-12 was tested against the primary fungal pathogen Histoplasma capsulatum, the opportunistic fungal pathogen, Aspergillus fumigatus, and the dermatophyte fungus Trichophyton rubrum. AR-12 effectively prevents growth of all three fungi at low concentrations (e.g., 8-40 μM). In another aspect, growth of all three fungi can be inhibited in part at concentrations at least about 3 μM. In contrast to the current fungistatic antifungal drugs, treatment with AR-12 led to killing of yeast and mycelia (e.g., Histoplasma capsulatum and Aspergillus fumigatus, respectively).

Further aspects described herein provide methods of inhibiting fungal growth in a host infected with one or more fungi selected from the group consisting of Paecilomyces, Rhizopus, Fusarium, Scedosporium, Lomentospora, Apophysomyces, Coccidioides, Blastomyces, non-albicans Candida, and Pneumocytis, comprising administering AR-12 to the host in an amount sufficient to achieve a concentration in the host to inhibit fungal growth in the host by about 100%. In these aspects, inventors utilized the non-clinical and pre-clinical services program offered by the National Institutes of Allergy and Infectious Diseases.

As used herein, the term AR-12, refers to (C₂₆H₁₉F₃N₄O and 2-amino-N-(4-(5-(phenanthren-2-yl)-3-(trifluoromethyl)-1H-pyrazol-1-yl)phenyl)acetamide)), having the following structure:

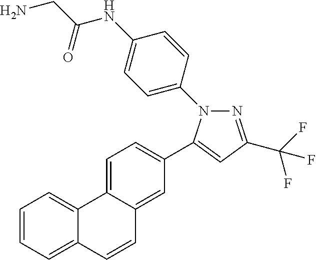 [SMILES: NCC(=O)Nc1ccc(-n2nc(C(F)(F)F)cc2-c2ccc3c(ccc4ccccc43)c2)cc1]

The term “AR-12” also includes, for example, analogs of AR-12 (e.g., the compounds described in U.S. Pat. Nos. 7,576,116, 8,546,441, 8,541,460, 8,039,502, and 8,080,574 hereby incorporated by reference in their entirety).

AR-12 Effects on the Respiratory Fungal Pathogen Histoplasma Capsulatum.

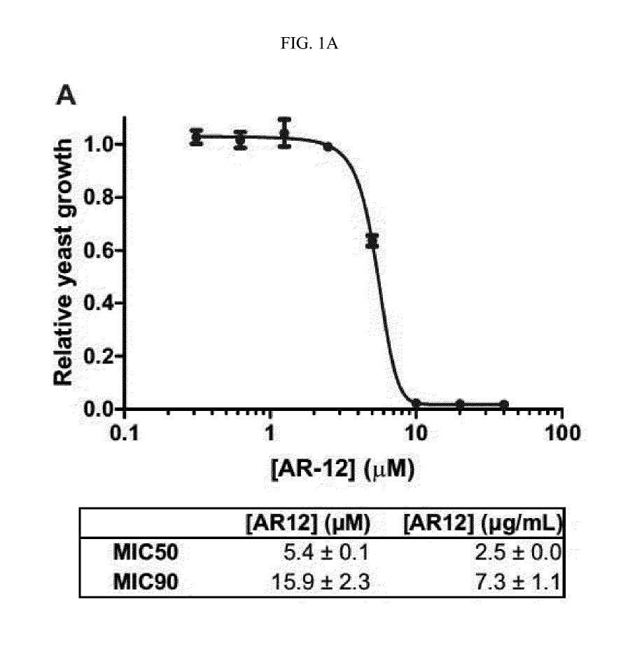

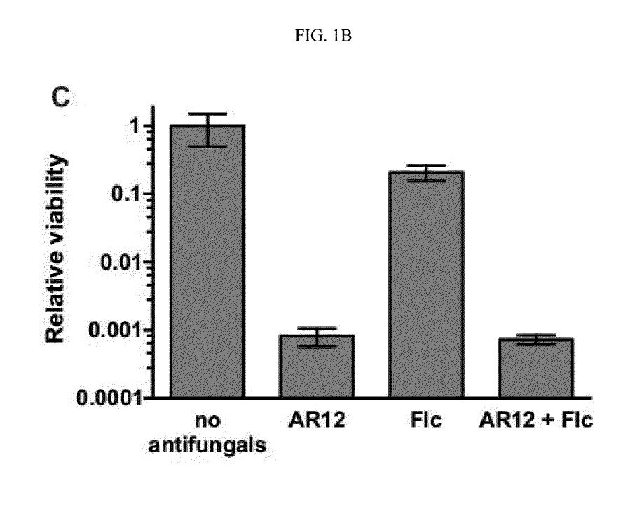

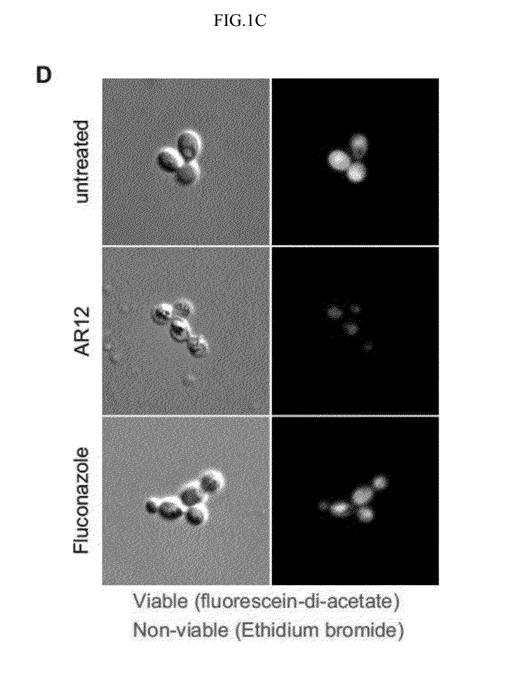

FIG. 1A shows an exemplary dose-response curve for the growth of Histoplasma capsulatum after treatment with AR-12. Minimal inhibitory concentrations (MICs) of AR-12 were determined from linear regression of the dose-response curve. AR-12 concentrations of 5.4 μM and 15.9 μM resulted in 50% and 90% inhibition of Histoplasma capsulatum yeast growth, respectively.

FIG. 1B illustrates the viability of Histoplasma capsulatum after treatment with AR-12, fluconazole, or a combination. Viability tests of Histoplasma capsulatum yeasts following 24-hour treatment with AR-12, fluconazole (Flc), or combination of AR-12 and fluconazole shows that AR-12 treatment reduces fungal viability about 1000-fold, whereas fluconazole did not significantly reduce fungal viability. In this aspect, viability was measured by growing AR-12-treated fungal cells in the absence of drug to see how many viable cells remained.

FIG. 1C shows viability staining of Histoplasma capsulatum yeasts following antifungal drug treatment. Visualization of Histoplasma capsulatum yeasts following 24-hour treatment with AR-12 or with fluconazole shows AR-12 treatment results in loss of yeast viability (indicated by ethidium bromide staining; red) whereas fluconazole treatment alone is only fungistatic leaving yeasts arrested in growth but still viable (indicated by fluorescein staining; green).

AR-12 Effects on the Opportunistic Fungal Pathogen Aspergillus fumigatus

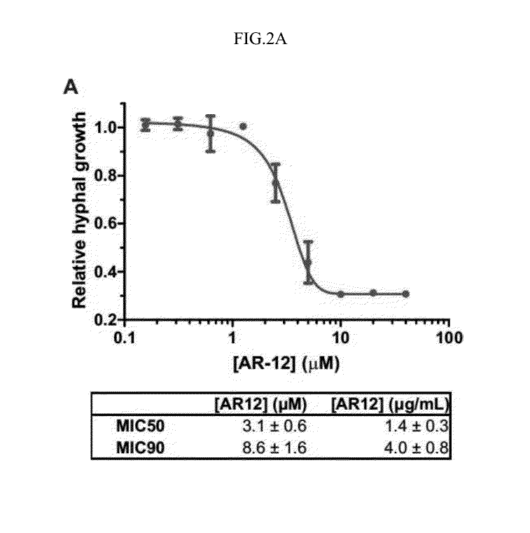

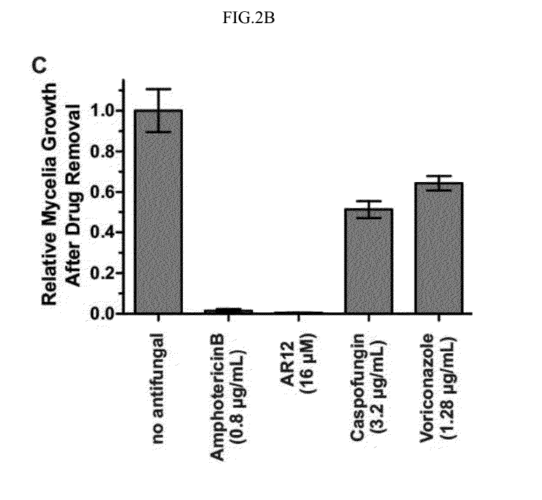

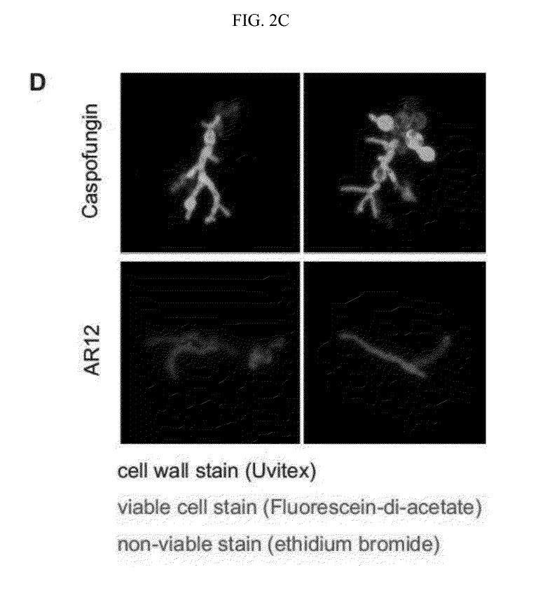

FIG. 2A is an exemplary dose-response growth curve for AR-12 treated Aspergillus fumigatus mycelia. Minimal inhibitory concentrations (MICs) of AR-12 were determined from linear regression of the dose-response curve. In this aspect, AR-12 concentrations of 3.1 μM and 8.6 μM result in 50% and 90% inhibition of Aspergillus fumigatus mycelia growth, respectively.

FIG. 2B is a graph illustrating relative mycelia growth following treatment with antifungal agents. Recovery of mycelia growth after removal of antifungal drugs shows no viable Aspergillus fumigatus mycelia after treatment with AR-12 and amphotericin B but treatment with the fungistatic drugs caspofungin and voriconazole leaves mycelia viable. Aspergillus fumigatus mycelia were treated for 12 hours after which the drugs were removed and the mycelia incubated for an additional 24 hours before measuring mycelia growth by metabolic reduction of the colorimetric substrate MTT (3-(4,5-dimethylthiazol-2-yl)-2,5-diphenyltetrazolium bromide) to its formazan(5-(4,5-dimethylthiazol-2yl)-1,3 -diphenylformazan).

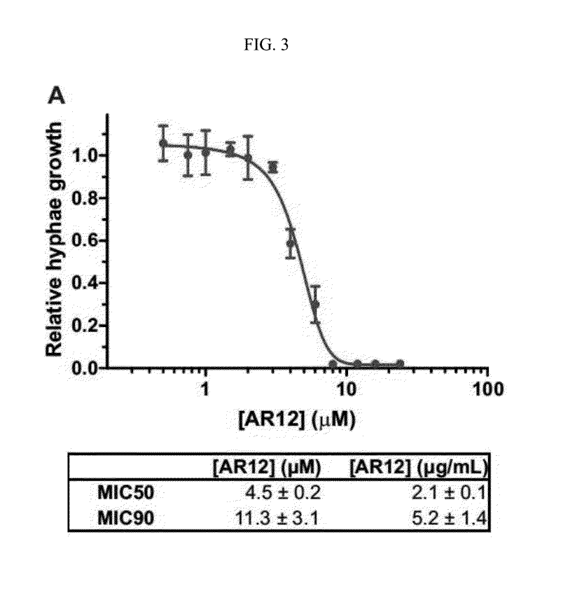

FIG. 3 is an exemplary dose-response curve for AR-12 on the growth of Trichophyton rubrum mycelia. Minimal inhibitory concentrations (MICs) of AR-12 were determined from linear regression of the dose-response curve. AR-12 concentrations of 4.5 μM and 11.3 μM result in 50% and 90% inhibition of Trichophyton rubrum mycelia growth, respectively.

Further aspects provide administering one or more additional compounds to the host (e.g., clotrimazole, econazole, ketoconazole, miconazole, tioconazole, fluconazole, posaconazole, itraconazole, voriconazole, isavuconazonium, terbinafine, nystatin, amorolfine, griseofulvin, caspofungin, micafungin, anidulafungin, tevaborole, efinaconazole, amphotericin B deoxycholate, and liposomal amphotericin B.

Yet additional aspects provide method of inhibiting fungal growth in a host infected with a fungus, comprising administering AR-12 to the host at a concentration between about 1 μg/ml to about 100 μg/ml for at least about 24 hours. AR-12 can also be administered to the host for at least about 48 hours or at least 72 hours or longer.

Further aspects provide methods of inhibiting fungal growth in a host infected with Pneumocytis comprising administering AR-12 to the host in an amount sufficient to achieve a concentration in the host to reduce fungal growth in the host by about 50%. In one aspect, AR-12 is administered at a concentration of about 4.82 μg/ml.

In another aspect, the Pneumocytis is Pneumocytis carinii. In this aspect, AR-12 can be administered in an amount sufficient to achieve a concentration of about 1.78 μg/ml in the host. In another aspect, the Pneumocytis is Pneumocytis marina. This aspect can further comprise administering one or more additional compounds to the host (e.g., clotrimazole, econazole, ketoconazole, miconazole, tioconazole, fluconazole, posaconazole, itraconazole, voriconazole, isavuconazonium, terbinafine, nystatin, amorolfine, griseofulvin, caspofungin, micafungin, anidulafungin, tevaborole, efinaconazole, amphotericin B deoxycholate, and liposomal amphotericin B).

In one aspect, AR-12 was tested against Paecilomyces, Rhizopus, Fusarium, Scedosporium, Lomentospora, Apophysomyces, Coccidioides, Candida, Cryptococcus, and Blastomyces. In another aspect, AR-12 was tested against C. parapsilosis, C. krusei, C. glabrata, C. guilliermondii, and C. neoformans. AR-12 effectively prevents growth of these fungi at concentrations ranging from 1 μg/ml to about 100 μg/ml or 1 to about 100 μg/ml. These data further demonstrate that AR-12 effectively prevents growth of non-albicans Candida species including, but not limited to, C. parapsilosis, C. krusei, C. glabrata, and C. guilliermondii.

In another aspect, growth of Pneumocytis fungi can be inhibited in part at concentrations at least about 1 μg/ml to about 100 μg/ml. In this aspect, AR-12 can be administered from 1 to about 24 hours, 24 to about 48 hours, 48 hours to about 72 hours, or 72 hours to 192 hours. In yet another aspect, AR-12 is administered in an amount of at least 1 μg/ml for at least 72 hours.

As shown in Table 1 below, AR-12, along with positive controls Posaconazole (POS) and Voriconazole (VORI), were provided to the indicated fungal isolate using the CLSI M38-A2 methodology (-e.g., M38-A2, Reference Method for Broth Dilution Antifungal Susceptibility Testing of Filamentous Fungi; Approved Standard—Second Edition, Clinical Laboratory Standards Institute, April 2008)) to calculate the Minimal Inhibitory Concentration (MIC). The MIC (concentration in gg/m1 for the amount of the drug required to kill 50% of the indicated fungi and the amount required to kill 100% of the fungi) for the indicated fungi and strain is provided. The time of exposure for each fungi is also provided below. The term “ND” means “Not Determined.” MICs for voriconazole (VORI) and posaconazole (POS) against Rhizopus oryzae & Apophysomyces (24 hours), Coccidioides immitis, Coccidioides posadasii, Fusarium oxysporum, Fusarium solani & Lomentospora prolificans (48 hours), and Scedosporium apiospermum (72 hours) were determined as 100% growth inhibition compared to growth controls. Blastomyces dermatitidis was tested using macrodilution methods (e.g., National Committee for Clinical Laboratory Standards (Document M27-P)), and read as 80% inhibition of growth at 96 hours.

TABLE 1

| | | AR-12 | AR-12 | POS | VORI |
|---|---|---|---|---|---|
| Isolates | | 50% | 100% | 100% | 100% |
| Paeceilomyces variotii | QC | 4 | 4 | 0.25 | 0.125 |
| Rhizopus oryze | RO1 | 4 | 4 | 1 | ND |
| Rhizopus oryze | RO2 | 4 | 4 | 1 | ND |
| Rhizopus oryze | RO3 | 4 | 4 | 1 | ND |
| Fusarium oxysporum | FO1 | 4 | 4 | ND | 16 |
| Fusarium oxysporum | FO2 | 4 | 4 | ND | 16 |
| Fusarium solani | FS1 | 4 | 4 | ND | 8 |
| Scedosporium | SA1 | 2 | 2 | ND | 0.25 |
| apiospermum | | | | | |
| Scedosporium | SA2 | 1 | 2 | 2 | 0.125 |
| apiospermum | | | | | |
| Lomentospora prolificans | LP1 | 2 | 2 | 0.5 | 16 |
| Apophysomyces | A1 | 2 | >16 | ND | ND |
| Apophysomyces | A2 | 2 | >16 | ND | ND |
| Coccidioides immitis/ | Cocci1 | 1 | 1 | ND | 0.25 |
| posadasii | | | | | |
| Coccidioides immitis/ | Cocci2 | 1 | 1 | ND | 0.25 |
| posadasii | | | | | |
| Coccidioides immitis/ | Cocci3 | 2 | 2 | ND | 0.25 |
| posadasii | | | | | |
| Blastomyces dermatitidis | BD1 | 2 | 4 | ND | 0.125 |
| Blastomyces dermatitidis | BD2 | 1 | 2 | ND | ≦0.03 |
| Blastomyces dermatitidis | BD3 | 1 | 1 | ND | ≦0.03 |

As shown in Tables 2 and 3 below, AR-12 reduced the in vitro ATP activity of Pneumocystis carinii and Pneumocystis murina in both a time and dose dependent manner. In one aspect, AR-12 was received in one shipment of 10.23 mg and stored at 4° C. without exposure to light. Just prior to testing, the compound was solubilized in 100% DMSO for a 50mg/m1 stock solution. Serial dilutions of 100, 10, 1, and 0.1m/m1 were made in RPMI-1640 containing 20% horse serum, 1% MEM vitamin solution, 1% MEM NEAA, and 2,000 units/ml Pen-Strep. Negative controls were media alone and 10 μg/ml ampicillin. Positive control was 1 μg/ml pentamidine isethionate. AR-12 was tested for luciferin/luciferase reaction interference at the above concentrations, and was found to have no quenching effect.

Cryopreserved and characterized P. carinii (Pc) isolated from rat lung tissue and P. murina (Pm) isolated from mouse lung tissue were distributed into triplicate wells of 48-well plates with a final volume of 500 μl and a final concentration of 5×10⁷ nuclei/ml Pc and 5×106 Pm. Controls and AR-12 dilutions were added and incubated at 36° C., 5% CO₂. At 24, 48, and 72 hours, 10% of the well volume was removed and the ATP content was measured using Perkin Elmer ATP-liteM luciferin-luciferase assay. The luminescence generated by the ATP content of the samples was measured by a BMG PolarStar optima spectrophotometer. A sample of each group was examined microscopically on the final assay day to rule out the presence of bacteria.

Background luminescence was subtracted and triplicate well readings of duplicate assays were averaged. For each day's readings, % reduction in ATP for all groups was calculated: experimental—experimental/vehicle control ×100. 50% inhibitory concentration (IC50) was calculated in INSTAT linear regression program.

In another aspect, the 72-hour IC₅₀ for AR-12 against P. carinii was 4.82 μg/ml and 1.78 μg/ml for P. carinii and P. murina respectively.

TABLE 2

| Pm % Reduction in ATP/Vehicle | | | |
|---|---|---|---|
| Control | 24 hours | 48 hours | 72 hours |
| Ampicillin 10 μg/ml | 0 | 0 | 0 |
| Pent. 1 μg/ml | 74.18 | 97.06 | 97.47 |
| AR-12 100 μg/ml | 97.31 | 97.2 | 99.04 |
| AR-12 10 μg/ml | 0 | 16.68 | 59.81 |
| AR-12 1 μg/ml | 0 | 1.76 | 0.49 |
| IC50 μg/ml | 41.3 | 18.32 | 4.82 |

TABLE 3

| Pm % Reduction in ATP/Vehicle | | | |
|---|---|---|---|
| Control | 24 hours | 48 hours | 72 hours |
| Ampicillin 10 μg/ml | 0 | 0 | 0 |
| Pent. 1 μg/ml | 72.06 | 87.94 | 93.85 |
| AR-12 100 μg/ml | 83.6 | 97.76 | 98.09 |
| AR-12 10 μg/ml | 54.02 | 85.38 | 94.23 |
| AR-12 1 μg/ml | 3.51 | 5.76 | 15.48 |
| IC50 μg/ml | 9.69 | 2.77 | 1.78 |

Table 4 provides the MICs against Candida species including non-albicans Candida species and Cryptococcus neoformans read at 24 and 72 hours, respectively (Table 4). “FLU” refers to Fluconazole and “POS” and VOR” refer to Posaconazole and Voriconazole respectively.

TABLE 4

| | | AR-12 | AR-12 | AR-12 | AR-12 | AR-12 | AR-12 | FLU |
|---|---|---|---|---|---|---|---|---|
| | | 50% | 100% | 50% | 100% | 50% | 100% | 50% |
| Species | | 24 hrs | 24 hrs | 48 hrs | 48 hrs | 72 hrs | 72 hrs | 24/72 hrs |
| C. parapsilosis | QC | 2 | 2 | 2 | 4 | ND | ND | 1 |
| C. krusel | QC | 2 | 4 | 4 | 4 | ND | ND | 8 |
| C. glabrata | CG-1 | 4 | 4 | 4 | 4 | ND | ND | 16 |
| C. glabrata | CG-2 | 4 | 4 | 4 | 4 | ND | ND | 16 |
| C. glabrata | CG-3 | 4 | 4 | 4 | 4 | ND | ND | 32 |
| C. glabrata | CG-4 | 4 | 4 | 4 | 4 | ND | ND | 32 |
| C. glabrata | CG-5 | 4 | 4 | 4 | 4 | ND | ND | 16 |
| C. glabrata | CG-6 | 4 | 4 | 4 | 4 | ND | ND | 2 |
| C. glabrata | CG-7 | 2 | 4 | 4 | 4 | ND | ND | 2 |
| C. glabrata | CG-8 | 4 | 4 | 4 | 4 | ND | ND | 2 |
| C. glabrata | CG-9 | 2 | 4 | 4 | 4 | ND | ND | 2 |
| C. glabrata | CG-10 | 2 | 4 | 4 | 4 | ND | ND | 4 |
| C. guilliermondii | Cgu-1 | 2 | 2 | 4 | 4 | ND | ND | 8 |
| C. guilliermondii | Cgu-2 | 2 | 2 | 2 | 4 | ND | ND | 2 |
| C. guilliermondii | Cgu-3 | 2 | 2 | 2 | 4 | ND | ND | 1 |
| C. guilliermondii | Cgu-4 | 2 | 2 | 2 | 2 | ND | ND | 1 |
| C. guilliermondii | Cgu-5 | 4 | 4 | 4 | 4 | ND | ND | 2 |
| C. guilliermondii | Cgu-6 | 4 | 4 | 4 | 4 | ND | ND | 8 |
| C. guilliermondii | Cgu-7 | 2 | 2 | 4 | 4 | ND | ND | 4 |
| C. parapsilosis | CP-1 | 2 | 2 | 4 | 4 | ND | ND | 0.25 |
| C. parapsilosis | CP-2 | 2 | 4 | 4 | 4 | ND | ND | ≦0.125 |
| C. parapsilosis | CP-3 | 2 | 4 | 4 | 4 | ND | ND | 0.25 |
| C. parapsilosis | CP-4 | 2 | 4 | 4 | 4 | ND | ND | 0.25 |
| C. parapsilosis | CP-5 | 2 | 2 | 4 | 4 | ND | ND | ≦0.125 |
| C. parapsilosis | CP-6 | 2 | 4 | 4 | 4 | ND | ND | 16 |
| C. parapsilosis | CP-7 | 2 | 2 | 2 | 4 | ND | ND | 16 |
| C. neoformans | CN-1 | ND | ND | 2 | 4 | 4 | 4 | 4 |
| C. neoformans | CN-2 | ND | ND | 4 | 4 | 4 | 4 | 12 |
| C. neoformans | CN-3 | ND | ND | 4 | 4 | 4 | 4 | 64 |

As shown in Table 4, AR-12 has a significant growth-inhibitory effect against non-albicans Candida species including, but not limited to, C. parapsilosis, C. krusei, C. glabrata, and C. guilliermondii.

Table 5 provides the MICs against Rhizopus oryzae, Aspergillus & Fusarium and Scedosporium species read at 24, 48, and 72 hours respectively (Table 5).

TABLE 5

| | | AR-12 | AR-12 | AR-12 | AR-12 | VOR | POS |
|---|---|---|---|---|---|---|---|
| | | 50% | 100% | 50% | 100% | 100% | 100% |
| Species | | 24 hrs | 24 hrs | 48/72 hrs | 48/72 hrs | 48/72 hrs | 24 hrs |
| P. variotii | QC | 2 | 4 | 4 | 4 | 0.06 | 0.06 |
| Fusarium sp. | F-1 | 2 | 2 | 4 | 4 | 8 | ND |
| Fusarium sp. | F-2 | 2 | 2 | 4 | 4 | 8 | ND |
| Fusarium sp. | F-3 | 2 | 2 | 4 | 4 | >16 | ND |
| Scedosporium sp. | S-1 | ND | ND | 2 | 4 | 0.5 | ND |
| Scedosporium sp. | S-2 | 2 | 2 | 4 | 4 | 16 | ND |
| R. oryzae | R-1 | 4 | 4 | 4 | 4 | ND | 0.5 |
| R. oryzae | R-2 | 4 | 4 | 8 | 8 | ND | 0.5 |
| R. oryzae | R-3 | 4 | 4 | 4 | 4 | ND | 0.5 |
| R. oryzae | R-4 | 4 | 4 | 4 | 4 | ND | 0.125 |
| R. oryzae | R-5 | 2 | 4 | 4 | 4 | ND | 0.25 |
| R. oryzae | R-6 | 4 | 4 | 4 | 4 | ND | 0.125 |
| Apophysomyces | AE-1 | 2 | 4 | 4 | 4 | ND | 0.125 |
| Apophysomyces | AE-2 | 2 | 4 | 4 | 4 | ND | 1 |

MICs against Blastomyces dermatitidis and Coccidioides species were read at between 48-168 hours (Table 6).

TABLE 6

| | | AR-12 | AR-12 | VORI 100% |
|---|---|---|---|---|
| | | 50% | 100% | 50% |
| Species | | 72/192 hrs | 72/192 hrs | 72/192 hrs |
| B. dermatitidis | BD1 | 0.25 | 0.5 | ≦0.03 |
| B. dermatitidis | BD2 | 2 | 2 | 0.125 |
| B. dermatitidis | BD3 | 1 | 2 | 0.125 |
| Coccidioides sp. | COCCI-1 | 2 | 4 | 0.125 |
| Coccidioides sp. | COCCI-2 | 2 | 8 | 0.25 |
| Coccidioides sp. | COCCI-3 | 2 | 4 | 0.125 |

AR-12, as described herein, can be administered orally, parenterally (IV, IM, depot-IM, SQ, and depot-SQ), sublingually, intranasally (inhalation), intrathecally, topically, in the pulmonary system or airways (e.g., nebulization, aerosol) or rectally. Dosage forms known to those of skill in the art are suitable for delivery of AR-12 described herein.

AR-12 can be formulated into suitable pharmaceutical preparations such as creams, gels, suspensions, tablets, capsules, or elixirs for oral administration or in sterile solutions or suspensions for parenteral administration. AR-12 can be formulated into pharmaceutical compositions using techniques and procedures well-known in the art.

In one aspect, about 0.1 to 1000 mg, about 5 to about 100 mg, or about 10 to about 50 mg of the AR-12, or a physiologically acceptable salt or ester can be compounded with a physiologically acceptable vehicle, carrier, excipient, binder, preservative, pain reliever, stabilizer, flavor, etc., in a unit dosage form as called for by accepted pharmaceutical practice. The amount of active substance in compositions or preparations comprising AR-12 is such that a suitable dosage and concentration in a host in the range indicated is obtained.

In another aspect, the compositions can be formulated in a unit dosage form, each dosage containing from about 1 to about 1000 mg, about 1 to about 500 mg, or about 10 to about 100 mg of the active ingredient. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

In one aspect, AR-12 alone or AR-12 and one or more additional active or inert ingredients, is mixed with a suitable pharmaceutically acceptable carrier to form a composition. Upon mixing or addition of the compound(s), the resulting mixture may be a cream, gel, solution, suspension, emulsion, or the like. Liposomal suspensions may also be used as pharmaceutically acceptable carriers. These may be prepared according to methods known to those skilled in the art. The form of the resulting mixture depends upon a number of factors, including the intended mode of administration and the solubility of the compound in the selected carrier or vehicle. In one aspect, the effective concentration is sufficient for lessening or ameliorating at least one symptom of the disease, disorder, or condition treated and may be empirically determined.

Pharmaceutical carriers or vehicles suitable for administration of AR-12 described herein include any such carriers suitable for the particular mode of administration. In addition, the active materials can also be mixed with other active materials that do not impair the desired action, or with materials that supplement the desired action, or have another action. The compounds may be formulated as the sole pharmaceutically active ingredient in the composition or may be combined with other active ingredients.

In another aspect, if AR-12 exhibits insufficient solubility, methods for solubilizing may be used. Such methods are known and include, but are not limited to, using co-solvents such as dimethylsulfoxide (DMSO), using surfactants such as TWEEN, and dissolution in aqueous sodium bicarbonate. Derivatives of the compounds, such as salts or prodrugs, may also be used in formulating effective pharmaceutical compositions.

The concentration of the compound is effective for delivery of an amount upon administration that lessens or ameliorates at least one symptom of the disorder for which the compound is administered. Typically, the compositions are formulated for single dosage administration.

In another aspect, AR-12 as described herein may be prepared with carriers that protect them against rapid elimination from the body, such as time-release formulations or coatings. Such carriers include controlled release formulations, such as, but not limited to, microencapsulated delivery systems. The active compound can be included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo model systems for the treated disorder.

In another aspect, AR-12 and compositions described herein can be enclosed in multiple or single dose containers. The enclosed compounds and compositions can be provided in kits, for example, including component parts that can be assembled for use. For example, AR-12 in lyophilized form and a suitable diluent may be provided as separated components for combination prior to use. A kit may include AR-12 and a second therapeutic agent for co-administration. AR-12 and second therapeutic agent may be provided as separate component parts. A kit may include a plurality of containers, each container holding one or more unit dose of AR-12 described herein. In one aspect, the containers can be adapted for the desired mode of administration, including, but not limited to suspensions, tablets, gel capsules, sustained-release capsules, and the like for oral administration; depot products, pre-filled syringes, ampoules, vials, and the like for parenteral administration; and patches, medipads, gels, suspensions, creams, and the like for topical administration.

The concentration of AR-12 in the pharmaceutical composition will depend on absorption, inactivation, and excretion rates of the active compound, the dosage schedule, and amount administered as well as other factors known to those of skill in the art.

In another aspect, the active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

If oral administration is desired, the compound can be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

Oral compositions will generally include an inert diluent or an edible carrier and may be compressed into tablets or enclosed in gelatin capsules. For the purpose of oral therapeutic administration, the active compound or compounds can be incorporated with excipients and used in the form of tablets, capsules, or troches. Pharmaceutically compatible binding agents and adjuvant materials can be included as part of the composition.

The tablets, pills, capsules, troches, and the like can contain any of the following ingredients or compounds of a similar nature: a binder such as, but not limited to, gum tragacanth, acacia, corn starch, or gelatin; an excipient such as microcrystalline cellulose, starch, or lactose; a disintegrating agent such as, but not limited to, alginic acid and corn starch; a lubricant such as, but not limited to, magnesium stearate; a glidant, such as, but not limited to, colloidal silicon dioxide; a sweetening agent such as sucrose or saccharin; and a flavoring agent such as peppermint, methyl salicylate, or fruit flavoring.

When the dosage unit form is a capsule, it can contain, in addition to material of the above type, a liquid carrier such as a fatty oil. In addition, dosage unit forms can contain various other materials, which modify the physical form of the dosage unit, for example, coatings of sugar and other enteric agents. The compounds can also be administered as a component of an elixir, suspension, syrup, wafer, chewing gum or the like. A syrup may contain, in addition to the active compounds, sucrose as a sweetening agent and certain preservatives, dyes and colorings, and flavors.

The active materials can also be mixed with other active materials that do not impair the desired action, or with materials that supplement the desired action. AR-12 can be used, for example, in combination with an antibiotic, antifungal, antiviral, pain reliever, or cosmetic.

In one aspect, solutions or suspensions used for parenteral, intradermal, subcutaneous, inhalation, or topical application can include any of the following components: a sterile diluent such as water for injection, saline solution, fixed oil, a naturally occurring vegetable oil such as sesame oil, coconut oil, peanut oil, cottonseed oil, and the like, or a synthetic fatty vehicle such as ethyl oleate, and the like, alcohols, polyethylene glycol, glycerin, propylene glycol, or other synthetic solvent; antimicrobial agents such as benzyl alcohol and methyl parabens; antioxidants such as ascorbic acid and sodium bisulfate; chelating agents such as ethylenediaminetetraacetic acid (EDTA); buffers such as acetates, citrates, and phosphates; and agents for the adjustment of tonicity such as sodium chloride and dextrose. Parenteral preparations can be enclosed in ampoules, disposable syringes, or multiple dose vials made of glass, plastic, or other suitable material. Buffers, preservatives, antioxidants, and the like can be incorporated as required.

Where administered intravenously, intramuscularly, or intraperitoneally, suitable carriers include, but are not limited to, physiological saline, phosphate buffered saline (PBS), and solutions containing thickening and solubilizing agents such as glucose, polyethylene glycol, polypropyleneglycol, ethanol, N-methylpyrrolidone, surfactants and mixtures thereof. Liposomal suspensions including tissue-targeted liposomes may also be suitable as pharmaceutically acceptable carriers. These may be prepared according to methods known in the art.

In another aspect, AR-12 may be prepared with carriers that protect the compound against rapid elimination from the body, such as time-release formulations or coatings. Such carriers include controlled release formulations, such as, but not limited to, implants and microencapsulated delivery systems, and biodegradable, biocompatible polymers such as collagen, ethylene vinyl acetate, polyanhydrides, polyglycolic acid, polyorthoesters, polylactic acid, and the like. Methods for preparation of such formulations are known to those skilled in the art.

In yet another aspect, compounds employed in the methods of the disclosure may be administered enterally or parenterally. When administered orally, compounds employed in the methods of the disclosure can be administered in usual dosage forms for oral administration as is well known to those skilled in the art. These dosage forms include the usual solid unit dosage forms of tablets and capsules as well as liquid dosage forms such as solutions, suspensions, and elixirs. When the solid dosage forms are used, they can be of the sustained release type so that the compounds employed in the methods described herein need to be administered only once or twice daily.

The dosage forms can be administered to the patient 1, 2, 3, or 4 times daily. AR-12 as described herein can be administered either three or fewer times, or even once or twice daily or every other day.

The terms “therapeutically effective amount” and “therapeutically effective period of time” are used to denote treatments at dosages and for periods of time effective to reduce neoplastic cell growth. As noted above, such administration can be parenteral, oral, sublingual, transdermal, topical, intranasal, or intrarectal. In one aspect, when administered systemically, the therapeutic composition can be administered at a sufficient dosage to attain a blood level of the compounds of from about 0.1 μM to about 20 μM. For localized administration, much lower concentrations than this can be effective, and much higher concentrations may be tolerated. One of skill in the art will appreciate that such therapeutic effect resulting in a lower effective concentration of AR-12 may vary considerably depending on the tissue, organ, or the particular animal or patient to be treated. It is also understood that while a patient may be started at one dose, that dose may be varied overtime as the patient's condition changes.

It should be apparent to one skilled in the art that the exact dosage and frequency of administration will depend on the particular compounds employed in the methods of the disclosure administered, the particular condition being treated, the severity of the condition being treated, the age, weight, general physical condition of the particular patient, and other medication the individual may be taking as is well known to administering physicians who are skilled in this art.

Not every element described herein is required. Indeed, a person of skill in the art will find numerous additional uses of and variations to the methods described herein, which the inventors intend to be limited only by the claims. All references cited herein are incorporated by reference in their entirety.

### REFERENCES

1. Booth L, Roberts J L, Cruickshanks N, Grant S, Poklepovic A, Dent P. Regulation of OSU-03012 toxicity by ER stress proteins and ER stress-inducing drugs. Mol Cancer Ther. 2014 October; 13(10):2384-98. doi: 10.1158/1535-7163.MCT-14-0172. Epub 2014 Aug 7. PubMed PMID: 25103559; PubMed Central PMCID: PMC4185238.

2. Chabrier-Roselló Y, Gerik K J, Koselny K, DiDone L, Lodge J K, Krysan D J. Cryptococcus neoformans phosphoinositide-dependent kinase 1 (PDK1) ortholog is required for stress tolerance and survival in murine phagocytes. Eukaryot Cell. 2013 January; 12(1):12-22. doi: 10.1128/EC.00235-12. Epub 2012 Oct. 19. PubMed PMID: 23087368; PubMed Central PMCID: PMC3535849.

3. Baxter B K, DiDone L, Ogu D, Schor S, Krysan D J. Identification, in vitro activity and mode of action of phosphoinositide-dependent-1 kinase inhibitors as antifungal molecules. ACS Chem Biol. 2011 May 20; 6(5):502-10. doi: 10.1021/cb100399x. Epub 2011 Feb. 22. PubMed PMID: 21294551; PubMed Central PMCID:PMC3098953.

4. Chiu H C, Kulp S K, Soni S, Wang D, Gunn J S, Schlesinger L S, Chen C S. Eradication of intracellular Salmonella enterica serovar Typhimurium with a small-molecule, host cell-directed agent. Antimicrob Agents Chemother. 2009 December; 53(12):5236-44. doi: 10.1128/AAC.00555-09. Epub 2009 Oct. 5. PubMed PMID: 19805568; PubMed Central PMCID: PMC27863 54.

5. Chiu H C, Yang J, Soni S, Kulp S K, Gunn J S, Schlesinger L S, Chen C S. Pharmacological exploitation of an off-target antibacterial effect of the cyclooxygenase-2 inhibitor celecoxib against Francisella tularensis. AntimicrobAgents Chemother. 2009 July; 53(7):2998-3002. doi: 10.1128/AAC.00048-09. Epub 2009Apr. 27. PubMed PMID: 19398640; PubMed Central PMCID: PMC2704645.

6. Brusselaers, et al.. Deep-seated Candida infections in the Intensive Care Unit, NETH J CRIT CARE, Vol. 15; No. 4, August 2011, pages 184-190.

7. Blot S et al. Is Candida really a threat in the ICU? Curr Opin Crit Care 2008; 14:600-604.

8. Blot S et al. Effects of nosocomial candidemia on outcomes of critically ill patients. Am J Med 2002; 113:480-485.

9. Darouiche R O. Candida in the ICU. Clin Chest Med 2009; 30:287-293, vi-vii.

10. Edwards J. et al, New York McGraw Hill Medical, Harrison's Principles of Internal Medicine, 18th Edition, 2012, 1651-1655.

What is claimed as new and desired to be protected by Letters Patent of the United States is:

1. A method of inhibiting fungal growth in a host infected with a fungus selected from the group consisting of Histoplasma capsulatum, Aspergillus fumigatus, and Trichophyton rubrum, comprising administering AR-12 to the host in an amount sufficient to reduce fungal growth in the host by about 90%.

2. The method of claim 1, wherein the fungal growth is reduced by about 50%.

3. The method of claim 2, wherein AR-12 is provided to the host in an amount sufficient to achieve a blood, tissue, or organ concentration between about 10 μM and 40 μM.

4. The method of claim 2, wherein AR-12 is provided to the host in an amount sufficient to achieve a blood, tissue or organ concentration between about 8 μM and 24 μM.

5. A method of inhibiting fungal growth in a host infected with a fungus, comprising administering AR-12 to the host in an amount sufficient to achieve a blood, tissue or organ concentration between about 3 μM and 8 μM, and wherein fungal growth is inhibited between about 10% and 50%.

6. A method of inhibiting fungal growth in a host infected with one or more fungi selected from the group consisting of Paecilomyces, Rhizopus, Fusarium, Scedosporium, Lomentospora, Apophysomyces, Coccidioides, Blastomyces, non-albicans Candida, and Pneumocytis, comprising administering AR-12 to the host in an amount sufficient to achieve a concentration in the host sufficient to inhibit fungal growth by about 100%.

7. The method of claim 6, wherein the fungal growth is reduced by about 50%.

8. The method of claim 6, wherein AR-12 is administered in an amount sufficient to achieve a concentration in the blood of the host between about 1 μg/ml to about 100 μg/ml, or an equivalent concentration in tissue or an organ.

9. The method of claim 7, wherein AR-12 is administered in amount sufficient to achieve a concentration in the blood of the host between about 1 μg/ml to about 16 μg/ml, or an equivalent concentration in tissue or an organ.

10. The method of claim 7, further comprising administering one or more additional compounds to the host, said one or more additional compounds selected from the group consisting of clotrimazole, econazole, ketoconazole, miconazole, tioconazole, fluconazole, posaconazole, itraconazole, voriconazole, isavuconazonium, terbinafine, nystatin, amorolfine, griseofulvin, caspofungin, micafungin, anidulafungin, tevaborole, efinaconazole, amphotericin B deoxycholate, and liposomal amphotericin B.

11. A method of inhibiting fungal growth in a host infected with a fungus, comprising:
administering AR-12 to the host in an amount sufficient to achieve a concentration in the blood of the host between about 1 μg/ml to about 100 μg/ml, or an equivalent concentration in tissue or an organ, for at least about 24 hours.

12. The method of claim 11, wherein AR-12 is administered for at least about 48 hours.

13. The method of claim 12, wherein AR-12 is administered for at least 72 hours.

14. A method of inhibiting fungal growth in a host infected with Pneumocytis comprising administering AR-12 to the host in an amount sufficient to achieve a concentration in the host to reduce fungal growth in the host by about 50%.

15. The method of claim 14, wherein AR-12 is administered to the host in an amount sufficient to achieve a concentration in the blood of about 4.82 μg/ml, or an equivalent concentration in a tissue or organ.

16. The method of claim 15, wherein the Pneumocytis is Pneumocytis carinii.

17. The method of claim 14, wherein AR-12 is administered to the host in an amount sufficient to achieve a concentration in the blood of about 1.78 μg/ml, or an equivalent concentration in a tissue or organ.

18. The method of claim 17, wherein the Pneumocytis is Pneumocytis marina.

19. The method of claim 11, further comprising administering one or more additional compounds to the host, said one or more additional compounds selected from the group consisting of clotrimazole, econazole, ketoconazole, miconazole, tioconazole, fluconazole, posaconazole, itraconazole, voriconazole, isavuconazonium, terbinafine, nystatin, amorolfine, griseofulvin, caspofungin, micafungin, anidulafungin, tevaborole, efinaconazole, amphotericin B deoxycholate, and liposomal amphotericin B.

20. A method of inhibiting fungal growth in a host infected with a non-albicans Candida fungus, comprising administering AR-12 to the host in an amount sufficient to achieve a concentration in the host to inhibit non-albicans Candida fungal growth by about 100%.

21. The method of claim 20, wherein the fungal growth is reduced by about 50%.

22. The method of claim 20, wherein AR-12 is administered in an amount sufficient to achieve a concentration in the host blood between about 1 μg/ml to about 100 μg/ml, or an equivalent concentration in a tissue or organ.

23. The method of claim 22, wherein AR-12 is administered in amount sufficient to achieve a concentration in the host blood between about 1 μg/ml to about 16 μg/ml, or an equivalent concentration in a tissue or organ.
